(19) United States
(12) Patent Application Publication
(10) Pub. No.: US 2009/0098248 A1
(43) Pub. Date: Apr. 16, 2009

(54) Novel Process for Enzymatic Acrylamide Reduction in Food Products

(76) Inventor: Lex De Boer, Wateringen (NL)

Correspondence Address: NIXON & VANDERHYE, PC, 901 NORTH GLEBE ROAD, 11TH FLOOR, ARLINGTON, VA, 22203, US

(21) Appl. No.: 11/920,428

(22) PCT Filed: May 29, 2006
(86) PCT No.: PCT/EP2006/062673

§ 371 Date: Nov. 15, 2007

(30) Foreign Application Priority Data
May 31, 2005 (EP) 05104683.7

(51) Int. Cl. A21D 8/04; C12N 9/82; C12N 9/18

(52) U.S. Cl. 426/61

(57) ABSTRACT

The present invention relates to a novel enzyme composition comprising asparaginase and at least one hydrolysing enzyme, the use of such composition to reduce acrylamide levels in food products and a method to produce food products involving at least one heating step, comprising adding:
a. asparaginase and
b. at least one hydrolyzing enzyme
to an intermediate form of said food product in said production process whereby the asparaginase and at least one hydrolyzing enzyme are added prior to said heating step in an amount that is effective in reducing the level of acrylamide of the food product in comparison to a food product whereto no asparaginase and hydrolyzing enzyme were added.

This invention relates to a novel enzyme composition suitable for use in a food preparation process in order to decrease acrylamide content in food products. The novel enzyme composition is especially suitable for use in baking industry. Recently, the occurrence of acrylamide in a number of food and oven prepared foods was published (Tareke et al. Chem. Res. Toxicol. 13, 517-522 (2000). Since acrylamide is considered as probably carcinogenic for animals and humans, this finding had resulted in world-wide concern. Further research revealed that considerable amounts of acrylamide are detectable in a variety of baked, fried and oven prepared common foods and it was demonstrated that the occurrence of acrylamide in food was the result of the baking process.

The official limit in the UK for acrylamide contamination in food products is set at 10 ppb (10 micrograms per kilogram) and the values presented above abundantly exceed this value for a lot of products, especially cereals, bread products and potato or corn based products.

A pathway for the formation of acrylamide from amino acids and reducing sugars as a result of the Maillard reaction has been proposed by Mottram et al. Nature 419:448 (2002). According to this hypothesis, acrylamide may be formed during the Maillard reaction. During baking and roasting, the Maillard reaction is mainly responsible for the color, smell and taste. A reaction associated with the Maillard is the Strecker degradation of amino acids and a pathway to acrylamide was proposed. The formation of acrylamide became detectable when the temperature exceeded 120° C., and the highest formation rate was observed at around 170° C. When asparagine and glucose were present, the highest levels of acrylamide could be observed, while glutamine and aspartic acid only resulted in trace quantities. The fact that acrylamide is formed mainly from asparagine (combined with reducing sugars) may explain the high levels acrylamide in oven-cooked or roasted plant products. Several plant raw materials are known to contain substantial levels of asparagine. In potatoes asparagine is the dominant free amino acid (940 mg/kg, corresponding with 40% of the total amino-acid content) and in wheat flour asparaginase is present as a level of about 167 mg/kg, corresponding with 14% of the total free amino acids pool (Belitz and Grosch in Food Chemistry—Springer New York, 1999). Therefore, in the interest of public health, there is an urgent need for food products that have substantially lower levels of acrylamide or, preferably, are devoid of it.

A variety of solutions to decrease the acrylamide content has been proposed, either by altering processing variables, e.g. temperature or duration of the heating step, or by chemically or enzymatically preventing the formation of acrylamide or by removing formed acrylamide. The present invention involves enzymatic decrease of formation of acrylamide.

Enzymatic routes to decrease the formation of acrylamide are amongst others the use of asparaginase to decrease the amount of asparagine in the food product, since asparagine is seen as an important precursor for acrylamide.

However, for some applications the use of asparaginase alone is not sufficient to decrease the acrylamide content of the food product to the desired level. Therefore, it is the object of the present invention to provide an enzyme composition resulting in an improved decrease of acrylamide levels in food prepared by use of the composition according to the invention.

The objective of the present invention is reached by providing an enzyme composition comprising asparaginase and at least one hydrolyzing enzyme.

Surprisingly, it was found that the addition of at least one hydrolyzing enzyme together with asparaginase results in a synergetic effect with respect to decrease acrylamide levels in food prepared with this enzyme composition.

An enzyme composition comprising asparaginase and an enzyme capable of oxidizing the reducing sugars is disclosed in WO 2004/032648 as is in line with the teaching that acrylamide is formed by the reaction between asparagine and reducing sugars.

However, the enzyme composition according to the present invention increases the amount of reducing sugars, but still reaches a dramatic decrease in the acrylamide level of the food product, even lower than when only asparaginase would have been added.

Any asparaginase (EC 3.5.1.1) available can be used in the present invention. Suitable asparaginase (E.C. 3.5.1.1) can be obtained from various sources, such as for Example from plants, animals and microorganisms. Examples of suitable microorganisms are Escheria, Erwinia, Streptomyces, Pseudomonas, Aspergillus and Baccillus species. Examples of suitable asparaginases can be found in WO03/083043 and WO2004/030468. A preferred asparaginase is the asparaginase having SEQ ID NO:3 or a functional equivalent thereof as described in WO04/030468 and which is disclosed herein by reference.

Any hydrolyzing enzyme (EC 3.x.x.x) can be suitable for the present invention. For the EC classification references as made herein the Recommended Enzyme Nomenclature (1992) of the IUBMB published by Academic Press Inc. (ISBN 0-12-227165-3) were used. X is herein used to indicate an integer.

However, preferably the hydrolyzing enzymes are used which belong to the group of carboxylic ester hydrolases (EC 3.1.1.x) or from the group of glycosidases hydrolyzing o-glycosyl compounds (EC 3.2.1.x.).

Examples of suitable carboxylic ester hydrolases are lipases (EC 3.1.1.3), pectin esterase (EC 3.1.1.11), galactolipase EC 3.1.1.26), phospholipase A1 (EC 3.1.1.32), phospholipase A2 (EC 3.1.1.4), lysophospholipase (EC 3.1.1.5).

Examples of preferred suitable hydrolysing o-glycosyl compounds are alpha-amylase (EC 3.2.1.1), beta-amylase (EC 3.2.1.2), pectinase (EC 3.2.1.15), cellulase (EC 3.2.1.4), xylanase (EC 3.2.1.32), arabinofuranosidase (EC 3.2.1.55), and glucanase (EC 3.2.1.6).

Also mixtures of hydrolyzing enzymes may be used in the composition according to the invention, including mixtures of carboxylic ester hydrolases with hydrolyzing o-glycosyl compounds. The person skilled in the art knows how to obtain the hydrolysing enzymes suitable for use in the invention.

In one preferred embodiment, asparaginase is combined with an enzyme selected from the group consisting of amylase, xylanase and lipase. These compositions are especially suitable for baking industry and might be part of a pre-mix.

In another preferred embodiment, asparaginase is combined with an enzyme which allows the mobilization of the asparaginase or the penetration of the asparaginase. These compositions are especially suitable when structurally intact cells of plant origin are present and an endogenous polymer of the plant matrix has to be hydrolysed.

In a second aspect of the invention, the invention relates to a novel process to reduce acrylamide content in food products. In one preferred embodiment, the food product is a baked product. In another preferred embodiment, the food product is a deep-fried product. In yet another preferred embodiment, the food product is a roasted or toasted product, in particular a roasted or toasted dough or bread.

The process for the production of a food product involving at least one heating step comprises adding asparaginase and at least one hydrolyzing enzyme to an intermediate form of said food product in said production process whereby the asparaginase and at least one hydrolyzing enzyme are added prior to said heating step in an amount that is effective in reducing the level of acrylamide of the food product in comparison to a food product whereto no asparaginase and hydrolyzing enzyme were added.

The asparaginase and at least one hydrolyzing enzyme can be added separately or in a composition, preferably in a composition according to the invention. Preferably, the composition is added to the food production process in an amount that the acrylamide content of the food product produced in the presence of the enzyme composition according to the invention is decreased relative to a food product produced without either one of the components in the composition according to the invention.

More preferably, the composition is added to the food production process in an amount that the acrylamide content of the food product produced in the presence of the enzyme is reduced by at least 10%, 15%, 20%, 25% or 30%, preferably by at least 35%, 40%, 45%, 50%, 55%, 60%, 65% or 70%, more preferably by at least 80%, 85% or 90%, most preferably by at least 95%, 97%, 98% or 99% as compared to food produced in the presence of asparaginase and in the absence of the hydrolyzing enzyme. For the asparaginase and the hydrolyzing enzymes to be used in the method according to the invention, the same preferences are to be considered as described above.

An intermediate form of the food product is defined herein as any form that occurs during the production process prior to obtaining the final form of the food product, this includes parts of plants, but also a slice or a cut of a plant part. The intermediate form may comprise the individual raw materials used and/or processed form thereof. To give just two Examples, for the food product bread, the intermediate forms can comprise wheat, wheat flour, the initial mixture thereof with other bread ingredients, such as for Example water, salt, yeast and bread improving compositions, the mixed dough, the kneaded dough, the frozen dough, the leavened dough and the partially baked dough, For the food product shaped potato chips, the intermediate forms can comprise boiled potato, mashed potato, dried mashed potato and potato dough.

The food product may be made from at least one raw material that is of plant origin, for Example potato, tobacco, coffee, cocoa, rice, cereal, fruit. Examples of cereals are wheat, rye, corn, maize, barley, groats, buckwheat and oat. Wheat is here and hereafter intended to encompass all known species of the Triticum genus, for Example aestivum, durum and/or spelta. Also food products made from more than one raw material are included in the scope of this invention, for Example food products comprising both wheat (flour) and potato.

Examples of food products in which the process according to the invention can be suitable for are any flour based products—for Example bread, pastry, cake, pretzels, bagels, Dutch honey cake, cookies, gingerbread, gingercake and crispbread —, and any potato-based products—for Example French fries, pommes frites, potato chips, croqueftes—and any corn-base product—for Example corn bread, corn crisps and corn flakes.

A preferred production process is the baking of bread and other baked products from wheat flour and/or flours from other cereal origin. Another preferred production process is the deep-frying of potato chips from potato slices. Still another preferred production process is the deep-frying of corn crisps from extruded corn based dough.

Preferred heating steps are those at which at least a part of the intermediate food product, e.g. the surface of the food product, is exposed to temperatures at which acrylamide formation is promoted, e.g. 110° C. or higher, 120° C. or higher. The heating step in the process according to the invention may be carried out in ovens, for instance at a temperature between 180-220° C., such as for the baking of bread and other bakery products, or in oil such as the frying of potato chips, for Example at 160-190° C.

The invention is hereafter illustrated by the following non-limiting Examples.

### SHORT DESCRIPTION OF THE FIGURES

FIG. 1 The effect of 50 ppm asparaginase in several enzyme combinations on acrylamide levels in crusts of mini-batards prepared with leavening salts (in %). The acrylamide level of the enzyme combination without asparaginase was set at 100%.

FIG. 2 The effect of 50 ppm A. niger asparaginase in several enzyme combinations on acrylamide levels in crusts of mini-batards prepared with Mogul Brand Chapatti brown flour and baker's yeast. The acrylamide level of the enzyme combination without asparaginase was set at 100%.

FIG. 3 The effect of A. niger asparaginase in several enzyme combinations on acrylamide levels in crusts of mini-batards prepared with kolibri flour and baker's yeast. The acrylamide level of bread with asparaginase as the sole baking enzyme was set at 100%.

### Materials

TABLE 1
Used baking enzymes in the Examples

| Baking enzyme | Enzyme activity | Supplier |
|---|---|---|
| Bakezyme P500 | Alpha-amylase | DSM Food Specialties |
| Bakezyme HSP6000 | Xylanase | DSM Food Specialties |
| Bakezyme W | Glucanase/cellulase | DSM Food Specialties |
| Bakezyme XE | Cellulase | DSM Food Specialties |
| Bakezyme A | Alpha-L-arabinofuranoside | DSM Food Specialties |
| | arabinofuranohydrolase/ | |
| | Arabinofuranosidase A | |
| Lipopan F | Galactolipase/ | Novozymes A/S |
| | phospholipase A1/ | |
| | phospholipase A2/ | |
| | Lysophospholipase/lipase | |

### EXAMPLE 1

### Acrylamide Measurement

### Sample Pretreatment

600 mg dried and homogenized sample is extracted using 5 ml of milliQ water. 1 μg of internal standard ¹³C₃ acrylamide in solution (CIL) is added to the extract. After 10 minutes of centrifugation (6000 rpm), 3 ml of the upper layer is brought on an Extreluut-3BT column (Merck). Using 15 ml of ethylacetate, acrylamide is eluted from the column. Ethylacetate is evaporated under a gentle stream of nitrogen down to approximately 0.5 ml.

### Chromatographic Conditions

The ethylacetate solution is analysed using gas chromatography. Separation is obtained using a CP-Wax 57 (Varian) column (length 25 m, internal diameter 0.32 mm, film 1.2 μm) and helium as the carrier gas with a constant flow of 5.4 ml/min. Split-less injection of 3 μl is performed. Oven temperature is kept at 50° C. for 1 minute, after which the temperature is increased with 30° C./min towards 220° C. After 12 minutes of constant temperature of 220° C. the oven is cooled down and stabilized before next injection.

Detection is performed using on-line chemical ionization mass spectrometry in positive ion mode, using methane as ionization gas. The characteristic ions m/z 72 (acrylamide) and m/z 75 (¹³C₃ acrylamide) are monitored for quantification.

### Used Equipment

| | |
|---|---|
| GC: | HP6890 (Hewlet Packard) |
| MSD (mass selective detector): | HP5973 (Hewlet Packard) |

Amounts in ppm or ppb are based on the amount of flour, unless stated otherwise.

### EXAMPLE 2

### Effects of Baking Enzymes and Aspergillus niger Asparaginase on Acrylamide Formation in Mini-Batard Breads Prepared with Leavening Salts

Preparation of minibatards with leavening salts was done by mixing 200 g whole-wheat flour (Mogul Brand Chapafti brown flour, Mogul Lasu B.V. The Hague, Holland), 4 g salt, 68 ppm ascorbic acid, 2 g DKS (NaHCO₃)(Chem Proha, Chemiepartners B.V. Dordrecht, Holland), 2.7 g Sap 40 (Sodium acid pyrophosphate, E450) (Chemische Fabrik Budenheim KG, Budenheim, Germany) 1 g SSL (Sodium stearoyl lactylate)(Danisco, Denmark) 1 g GMS (glyceryl mono stearate, (Admul), Quest, Naarden, Holland) Amounts of baking enzymes to be tested are indicated in Table 1 (Lipopan F and Novamyl are obtainable from Novo, the other enzymes are obtainable from DSM-Gist). 226 ml water was added. Mixing took place in a pin mixer for 8 minutes and 45 seconds. The dough temperature was 27° C. Directly after mixing the dough is divided into two pieces of 150 g, rounded and proofed for 25 minutes in a proofing cabinet at 32° C. Hereafter the dough pieces were shaped and a final proof was performed at 32° C. for 100 minutes. The dough pieces were baked for 20 minutes at 225° C. The acrylamide in the crust was determined as is described in Example 1. The percentage acrylamide that was left in the asparaginase treated breads was calculated as follows:

$$\frac{\begin{matrix}acrylamide content \\ ( baking enzyme combination \times plus asparaginase )\end{matrix} }{\begin{matrix}acrylamide content \\ ( baking enzyme combination \times without asparaginase )\end{matrix} } \times 100 \%$$

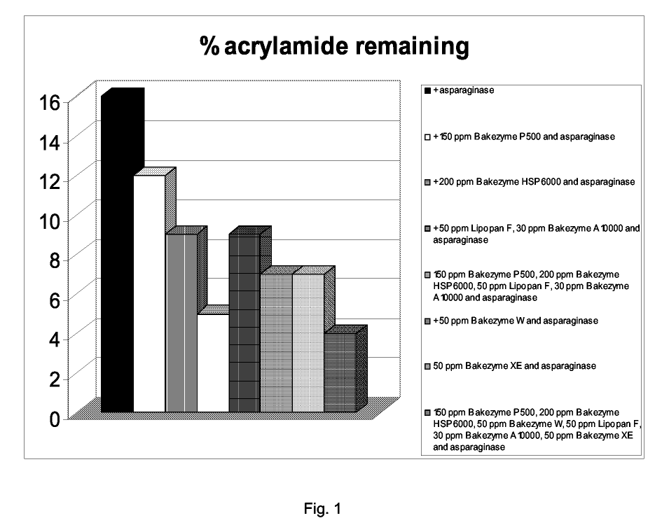

and is shown in Table 2 and FIG. 1 for several enzyme combinations. For Example, the percentage acrylamide remaining in bread treated with Bakezyme P500 and asparaginase was calculated by dividing the results from test no. 4 by the results from test no. 3 and multiplying this by 100%.

TABLE 2
Acrylamide in crusts of mini-batard breads prepared with leavening
salts and several baking enzymes as is indicated in the Example
and the effect of Aspergillus niger asparaginase on acrylamide levels.

| Test | | Dosage | Acrylamide | % acrylamide |
|---|---|---|---|---|
| no. | Baking Enzyme | (ppm) | content (ppb) | remaining |
| 1 | None | | 185 | 100 |
| 2 | Asparaginase | 50 | 30 | 16 |
| 3 | Bakezyme P500 | 150 | 143 | 100 |
| 4 | Bakezyme P500 | 150 | 17 | 12 |
| | Asparaginase | 50 | | |
| 5 | Bakezyme HSP6000 | 200 | 234 | 100 |
| 6 | Bakezyme HSP6000 | 200 | 21 | 9 |
| | Asparaginase | 50 | | |
| 7 | Lipopan F | 50 | 250 | 100 |
| | Bakezyme A10000 | 30 | | |
| 8 | Lipopan F | 50 | 13 | 5 |
| | Bakezyme A10000 | 30 | | |
| | Asparaginase | 50 | | |
| 9 | Bakezyme P500 | 150 | 279 | 100 |
| | Bakezyme HSP6000 | 200 | | |
| | Lipopan F | 50 | | |
| | bakezyme A10000 | 30 | | |
| 10 | Bakezyme P500 | 150 | 25 | 9 |
| | Bakezyme HSP6000 | 200 | | |
| | Lipopan F | 50 | | |
| | Bakezyme A10000 | 30 | | |
| | asparaginase | 50 | | |
| 11 | Bakezyme W | 50 | 263 | 100 |
| 12 | Bakezyme W | 50 | 19 | 7 |
| | asparaginase | 50 | | |
| 13 | Bakezyme XE | 50 | 228 | 100 |
| 14 | Bakezyme XE | 50 | 17 | 7 |
| | asparaginase | 50 | | |
| 15 | Bakezyme P500 | 150 | 464 | 100 |
| | Bakezyme HSP6000 | 200 | | |
| | Bakezyme W | 50 | | |
| | Lipopan F | 50 | | |
| | Bakezyme A10000 | 30 | | |
| | Bakezyme XE | 50 | | |
| 16 | Bakezyme P500 | 150 | 18 | 4 |
| | Bakezyme HSP6000 | 200 | | |
| | Bakezyme W | 50 | | |
| | Lipopan F | 50 | | |
| | Bakezyme A10000 | 30 | | |
| | Bakezyme XE | 50 | | |
| | asparaginase | 50 | | |

From Table 2 and FIG. 1 it can be concluded that addition of the baking enzymes Bakezyme® HSP6000, Lipopan® F, Bakezyme® A10000, Bakezyme® W, Bakezyme® XE and combinations thereof, will result in an increased level of acrylamide in the crust compared to a reference bread without baking enzymes. Addition of an appropriate amount of asparaginase to the dough will however result in a decreased level of acrylamide compared to the corresponding reference without asparaginase and even lower than a reference in which no baking enzymes were used.

### EXAMPLE 3

### Effects of Baking Enzymes and A. niger Asparaginase on Acrylamide Formation in the Mini-Batards Breads Prepared with Baking Yeast and Whole Wheat Flour

Preparation of mini-batard breads in a standard baking process was done by mixing 200 g of whole-wheat flour (Mogul Brand Chapatti brown flour) 4.6 g Koningsgist® yeast, 4 g salt, 68 ppm ascorbic acid and several enzymes and enzyme combinations as indicated in Table 2. 132 g water was added and mixing was performed in a pin mixer for 8 minutes and 45 seconds. The dough temperature was 27° C. Directly after mixing the dough was divided into two pieces of 150 g, rounded and proofed for 25 minutes in a proofing cabinet at 32° C. Hereafter, the dough pieces were shaped and a final proof was performed of 100 minutes at 32° C., the dough pieces were baked for 20 minutes at 225° C. The acrylamide in the crust was determined as is described in Example 1. The percentage acrylamide that was left in the asparaginase treated breads was calculated as is indicated in Example 2.

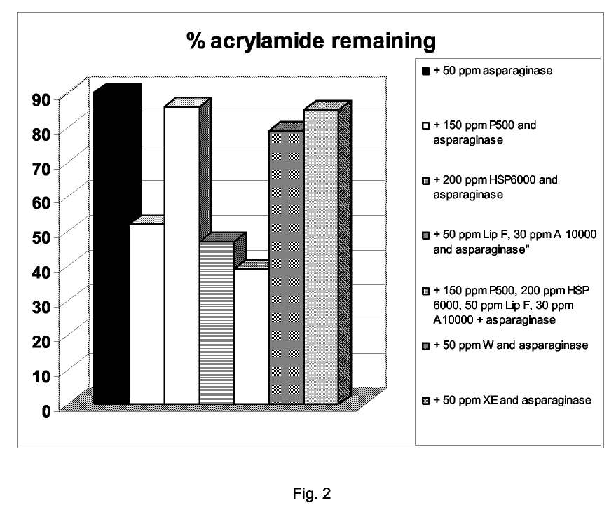

In Table 3 and FIG. 2 the effects of asparaginase are shown in several enzyme combinations.

TABLE 3
Acrylamide in crusts of mini-batard breads prepared with whole
wheat flour, yeast and several baking enzymes and the effect of
Aspergillus niger asparaginase on acrylamide levels.

| Test | | | Acrylamide | remaining % |
|---|---|---|---|---|
| no. | Baking Enzyme | Dosage (ppm) | content (ppb) | acrylamide |
| 1 | None | | 78 | 100 |
| 2 | asparaginase | 50 | 70 | 90 |
| 3 | Bakezyme P500 | 15 | 73 | 100 |
| 4 | Bakezyme P500 | 15 | 65 | 89 |
| | asparaginase | 50 | | |
| 5 | Bakezyme P500 | 150 | 94 | 100 |
| 6 | Bakezyme P500 | 150 | 49 | 52 |
| | Asparaginase | 50 | | |
| 7 | Bakezyme HSP6000 | 50 | 77 | 100 |
| 8 | Bakezyme HSP6000 | 50 | 67 | 87 |
| | Asparaginase | 50 | | |
| 9 | Bakezyme HSP6000 | 200 | 70 | 100 |
| 10 | Bakezyme HSP6000 | 200 | 60 | 86 |
| | Asparaginase | 50 | | |
| 11 | Lipopan F | 50 | 159 | 100 |
| | Bakezyme A10000 | 30 | | |
| 12 | Lipopan F | 50 | 74 | 7 |
| | Bakezyme A10000 | 50 | | |
| | Asparaginase | 50 | | |
| 13 | Bakezyme XE | 50 | 80 | 100 |
| 14 | Bakezyme XE | 50 | 68 | 85 |
| | Asparaginase | 50 | | |
| 15 | Bakezyme P500 | 150 | 257 | 100 |
| | Bakezyme HSP6000 | 200 | | |
| | Bakezyme A10000 | 30 | | |
| | Lipopan F | 50 | | |
| 16 | Bakezyme P500 | 150 | 100 | 39 |
| | Bakezyme HSP6000 | 200 | | |
| | Bakezyme A10000 | 30 | | |
| | Lipopan F | 50 | | |
| | Asparaginase | 50 | | |
| 17 | Bakezyme W | 50 | 90 | 100 |
| 18 | Bakezyme W | 50 | 71 | 79 |
| | Asparaginase | 50 | | |

In FIG. 2 the effects are presented of A. niger asparaginase in the presence of (combinations) of enzymes. Compared to the acrylamide level in crust of breads prepared with the mentioned an enzyme or enzyme combination, the relative and in some cases even the absolute acrylamide levels are lower when asparaginase is used in the presence of (combinations) of enzymes.

From Table 3 and FIG. 2 it can be concluded that addition of the baking enzymes Bakezyme P500, Bakezyme A10000, Bakezyme HSP6000, Lipopan F, Bakezyme W, Bakezyme XE and combinations thereof, will result in an increased level of acrylamide in the crust compared to a reference bread indifferent whether it is prepared with the leavening salt NaHCO₃ or yeast. Addition of an appropriate amount of asparaginase to the dough will however result in a decreased level of acrylamide compared to the corresponding reference without asparaginase and in some cases even lower than a reference in which no baking enzymes were used but where asparaginase was present.

### EXAMPLE 4

### Effects of Baking Enzymes and A. niger Asparaginase on Acrylamide Formation in the Mini-Batards Breads Prepared with Baking Yeast and Kolibri Flour

Preparation of mini-batard breads in a standard baking process was done by mixing 200 g of kolibri flour (Meneba) 4.6 g Koningsgist® yeast, 4 g salt, 68 ppm ascorbic acid and several enzymes and enzyme combinations as indicated in Table 2.114 g water was added and mixing was performed in a pin mixer for 6 minutes and 15 seconds. The dough temperature was 27° C. Directly after mixing the dough was divided into two pieces of 150 g, rounded and proofed for 25 minutes in a proofing cabinet at 32° C. Hereafter, the dough pieces were shaped and a final proof was performed of 100 minutes at 32° C., the dough pieces were baked for 20 minutes at 225° C. The acrylamide in the crust was determined as is described in Example 1. The percentage acrylamide that was left in the asparaginase treated breads was calculated as is indicated in Example 2.

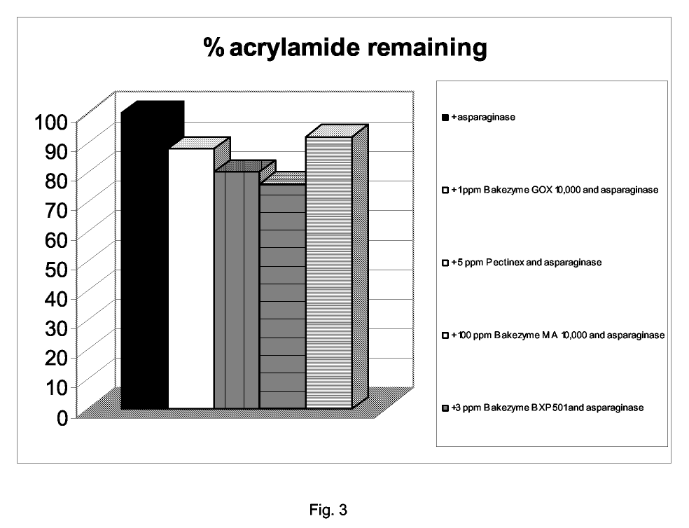

In Table 4 and FIG. 3 the effects of asparaginase are shown in several enzyme combinations.

TABLE 4
Acrylamide in crusts of mini-batard breads prepared kolibri
flour with yeast and several baking enzymes and the effect of
Aspergillus niger asparaginase on acrylamide levels.

| Test | | Dosage | Acrylamide | remaining % |
|---|---|---|---|---|
| no. | Baking Enzyme | (ppm) | content (ppb) | acrylamide |
| 1 | None | | 50 | 100 |
| 2 | asparaginase | 50 | 42 | 84 |
| 3 | Bakezyme GOX 10,000 | 1 | 40 | 100 |
| 4 | Bakezyme GOX 10,000 | 1 | 37 | 93 |
| | asparaginase | 50 | | |
| 5 | Pectinex* | 5 | 41 | 100 |
| 6 | Pectinex | 5 | 34 | 83 |
| | Asparaginase | 50 | | |
| 7 | Bakezyme MA 10,000 | 100 | 48 | 100 |
| 8 | Bakezyme MA 10,000 | 100 | 32 | 67 |
| | Asparaginase | 50 | | |
| 9 | Bakezyme BXP501 | 3 | 43 | 100 |
| 10 | Bakezyme BXP501 | 3 | 39 | 91 |
| | Asparaginase | 50 | | |

*Pectinex is derived from NOVO.

In FIG. 3 the effects are presented of A. niger asparaginase in the presence of (combinations) of enzymes. Compared to the acrylamide level in crust of breads prepared with the mentioned enzyme or enzyme combination, the absolute acrylamide levels are lower when asparaginase is used in the presence of (combinations) of enzymes. In some cases the relative amount of acrylamide that is left is higher as a result of the lower acrylamide content in the absence of the enzyme asparaginase. The absolute acrylamide level in the presence of the enzyme combination plus asparaginase is however lower than the reference.

From Table 4 and FIG. 3 it can be concluded that addition of the baking enzymes Bakezyme GOX 10,000, Bakezyme MA 10,000, Bakezyme BXP501 and Pectinex to a kolibri flour-based dough will result in a lower level of acrylamide in the crust when the enzyme or enzyme combination are combined with an appropriate amount of asparaginase, compared to a reference bread with asparaginase as the sole baking enzyme.

1. Enzymatic composition comprising:
a. asparaginase and;
b. at least one hydrolyzing enzyme.

2. Composition according to claim 1 wherein the hydrolyzing enzyme is a carboxylic ester hydrolase.

3. Composition according to claim 1 wherein the hydrolyzing enzyme is a glycosidase hydrolyzing o-glycosyl compound.

4. A food product having decreased acrylamide levels therein which comprises a composition according to claim 1.

5. Method process for the production of a food product involving at least one heating step, comprising adding:
a. asparaginase and
b. at least one hydrolyzing enzyme
to an intermediate form of said food product in said production process whereby the asparaginase and at least one hydrolyzing enzyme are added prior to said heating step in an amount that is effective in reducing the level of acrylamide of the food product in comparison to a food product whereto no asparaginase and hydrolyzing enzyme were added

6. Method according to claim 5 whereby components a. and b. are added in a single composition.

7. Method according to claim 5, whereby the food product is a baked product.

8. Method according to claim 5, whereby the food product is a deep fried, toasted or roasted product.

9. Method according to claim 5, wherein said intermediate form of said food product is a dough.

10. Method according to claim 5, wherein said food product is made from at least one raw material of plant origin.

11. A food product obtained by the the method according to claim 5.
